(19) United States
(12) Patent Application Publication
(10) Pub. No.: US 2023/0051263 A1
(43) Pub. Date: Feb. 16, 2023

(54) DEVICE FOR RAPID AND QUANTITATIVE DETECTION OF DRUGS OF ABUSE IN SWEAT

(71) Applicants: THE REGENTS OF THE UNIVERSITY OF MICHIGAN, Ann Arbor, MI (US); ARBORSENSE INC., Ann Arbor, MI (US); OPTOFLUIDIC BIOASSAY, LLC, Ann Arbor, MI (US)

(72) Inventors: Xudong FAN, Saline, MI (US); Wen XUE, Ann Arbor, MI (US); Xiaotian TAN, Ann Arbor, MI (US); Mark A. ILGEN, Ann Arbor, MI (US); Girish KULKARNI, Ann Arbor, MI (US); Maung Kyaw KHAING OO, Ann Arbor, MI (US)

(73) Assignees: THE REGENTS OF THE UNIVERSITY OF MICHIGAN, Ann Arbor, MI (US); ARBORSENSE INC., Ann Arbor, MI (US); OPTOFLUIDIC BIOASSAY, LLC, Ann Arbor, MI (US)

(21) Appl. No.: 17/791,790

(22) PCT Filed: Jan. 11, 2021
(86) PCT No.: PCT/US2021/012949

§ 371 Date: Jul. 8, 2022

Related U.S. Application Data
(60) Provisional application No. 62/958,957, filed on Jan. 9, 2020.

(51) Int. Cl. G01N 33/94; B01L 3/00

(52) U.S. Cl. CPC G01N 33/94; B01L 3/50855; B01L 2200/16; B01L 2300/16; B01L 3/50857; G01N 2021/6439

(57) ABSTRACT

An automated microfluidic bioreactor device and methods to rapidly detect drugs of abuse from human sweat are provided. The bioreactor can perform either single-plexed measurements (detecting only one target analyte at a time) or multiplexed measurement (detecting multiple analytes simultaneously). The bioreactor device has a cartridge comprising a capillary array that employs competitive enzyme-linked immunosorbent assay (ELISA) to detect the presence of various drugs or metabolite compounds. For example, four common drugs, methadone, methamphetamine, amphetamine, and tetrahydrocannabinol, were detected rapidly and quantitatively in about 16 minutes with a low sweat sample volume (about 4 μL per analyte) and a large dynamic range (methadone: 0.0016 ng/mL-1 ng/mL; METH: 0.016 ng/mL-25 ng/mL; amphetamine: 0.005 ng/mL-10 ng/mL; THC: 0.02 ng/mL-1000 ng/mL).

### CROSS-REFERENCE TO RELATED APPLICATIONS

This application is a PCT International Application of U.S. Patent Application No. 62/958,957 filed on Jan. 9, 2020. The entire disclosure of the above application is incorporated herein by reference.

### FIELD

The present disclosure relates to a device and a method to rapidly detect drugs of abuse from human sweat. More specifically, the present disclosure provides a method of detecting one or more drugs in a sample of sweat from a subject and an automated microfluidic bioreactor for detecting one or more drugs in a sample of sweat from a subject.

### BACKGROUND

This section provides background information related to the present disclosure which is not necessarily prior art.

The use and abuse of potentially addictive substances has become a national and global crisis. As reported by the 2018 National Survey on Drug Use and Health by the Substance Abuse and Mental Health Services Administration (SAMHSA) of United States (U.S.) Department of Health and Human Services (HHS), approximately 25 million people in the U.S. alone had used drugs of abuse in the previous month. The social (greater than about 70,000 deaths every year) and economic costs ($200 billion annually in healthcare and treatment) of drug use and abuse are immense. The likely legalization of cannabis across the U.S. will only worsen this crisis. Thus, rapid, sensitive, and on-site detection of drugs of abuse is essential for hospital testing, workplace drug-use screening, roadside testing, and patient monitoring in drug rehabilitation centers. To be particularly useful, results of drug detection would be quantitative and provide information, not just about the presence or absence of the substance, but also about the level of the substance that is present in the system.

Biological sources for drug analysis include blood, urine, saliva, hair, sweat, and exhaled breath. Normally, blood analysis provides an accurate approach to tracking drug dosage for hours, because the parent compounds of the drugs can be found in blood. However, it is invasive and can cause pain and intense stress in certain patients. Urine can also be tested for the parent compounds, as well as metabolites of various drugs. One of the main benefits of the urine test is that it is non-invasive and a specimen can be collected within minutes. Hair testing is the best long-term drug monitoring method, because hair grows slowly and drugs can be detected months or even years after drug is ingested. However, the processing time for hair testing is much longer than blood or urine testing. Alternatively, sweat analysis shows great advantages in drug detection as a non-invasive diagnosis method. It can record drug abuse history over a long period (up to 14 days), depending on the specific substance being examined. The distinctive characteristics of secretion, accessibility, and abundance in biomolecules make sweat an ideal candidate for drug monitoring.

The most widely used method in sweat testing is to use a sweat patch, which accumulates drugs or metabolites via a semipermeable membrane. The membrane is subsequently sent to a lab for analysis. However, the whole sweat collection and assay process takes 7 to 10 days. Recent research on imaging of fingerprint sweat using magnetic particles was shown to provide a fast and on-site approach to detecting drugs and user identification simultaneously. However, only qualitative or binary information (i.e., yes/no) can be obtained. Wearable sensors provide another way to detect drugs in sweat. Those sensors usually include electrical components for signal transduction and data transmission. They can be portable, fast, and convenient. The main challenges for wearable sensors are limited sensitivities and different detection mechanisms may be needed for different drugs in order to achieve detection specificity.

In contrast, conventional analytical techniques, such as gas chromatography and liquid chromatography coupled with mass spectrometry, can provide superior sensitivity and selectivity for drug detection in sweat, but they require expensive instruments and tedious sample pretreatment. Immunoassay based on 96-well plates combined with colorimetry, fluorescence, and chemiluminescence has been employed as one of the primary tools for quantitative detection of drugs and the associated metabolites. Long assay time and high cost make such assays more suitable for laboratory use (for example, a methadone detection kit from Neogen Corporation retails for $230 dollars per 96-well plate and takes longer than 1.5 hours for analyte quantification).

It would be desirable to develop additional methods of rapid, sensitive, and quantitative on-site detection of drugs of abuse in sweat, especially for multi-drug analysis.

### SUMMARY

This section provides a general summary of the disclosure, and is not a comprehensive disclosure of its full scope or all of its features.

In certain aspects, the present disclosure contemplates methods and bioreactor microfluidic devices for detecting one or more drugs in a sample of sweat from a subject. In one variation, the method comprises measuring a first luminescence value of a test sample in a bioreactor microfluidic device having a test capillary with a surface comprising at least one antibody. The test sample comprises (i) the sample of sweat from the subject that optionally comprises a target analyte capable of binding with the at least one antibody and (ii) one or more competitive conjugates comprising a luminescent moiety and an analyte capable of binding with the at least one antibody. The measuring occurs by contacting the test sample with the surface to permit competitive binding of the at least one antibody with the one or more competitive conjugates or the target analyte. The measuring occurs within 30 minutes of introducing the test sample into the test capillary. The method also comprises comparing the first luminescence value with a second luminescence value of a comparative capillary in the bioreactor microfluidic device having a comparative surface comprising the at least one antibody contacted with a comparative sample having the one or more competitive conjugates, but lacking the target analyte, to determine an amount of the target analyte present in the test sample.

In one aspect, prior to the measuring, the method further comprises forming a test sample by mixing the one or more competitive conjugates comprising the luminescent moiety and the analyte capable of binding with at least one antibody into the sample of sweat obtained from the subject that optionally comprises the target analyte capable of binding with the at least one antibody.

In one aspect, the method further comprises collecting the sample of sweat from the subject. The sample of sweat has a total volume of less than or equal to about 4 μL.

In one aspect, the test sample comprises a plurality of distinct competitive conjugates respectively capable of binding with a plurality of distinct antibodies and optionally a plurality of distinct target analytes respectively capable of binding with the plurality of distinct antibodies. The surface of the capillary comprises the plurality of distinct antibodies for competitively binding with the plurality of distinct competitive conjugates or the plurality of target analytes.

In one aspect, the bioreactor microfluidic device comprises a plurality of capillaries and the introducing comprises selectively introducing the test sample into the plurality of capillaries.

In one aspect, at least a portion of the bioreactor microfluidic device is disposable.

In one aspect, a duration of the measuring is less than or equal to about 20 minutes.

In one aspect, the analyte and the target analyte are the same compound, derivative, or metabolite selected from the group consisting of: cannabinoids, tetrahydrocannabinol (THC), methadone, amphetamine, methamphetamine (METH), 3,4-methylenedioxymethamphetamine, methylenedioxymethamphetamine (MDMA), methylenedioxyamphetamine (MDA), cocaine, benzoylecgonine (BZE), opiates, heroin, codeine, diacetylmorphine, 6-acetylmorphine, morphine, hydrocodone, hydromorphone, oxycodone, oxymorphone, fentanyl, phencyclidine (PCP), benzodiazepines, diazepam, alprazolam, anabolic steroids, performance enhancing compounds, cathinones, ethanol, nicotine, caffeine, and combinations thereof.

In one aspect, a detection limit of the bioreactor microfluidic device for the target analyte is less than or equal to about 150 pg/mL.

The present disclosure also provides an automated microfluidic bioreactor for detecting one or more drugs in a sample of sweat from a subject. The bioreactor in certain aspects comprises a cartridge including a plurality of capillaries. Each capillary comprises at least one opening and a surface having at least one antibody disposed thereon for participating in a competitive chemiluminescent enzyme-linked immunosorbent assay (ELISA). A portion of the plurality of capillaries is configured to receive a test sample comprising sweat from the subject, one or more competitive conjugates comprising a luminescent moiety and one or more analytes, and optionally one or more target analytes. The one or more analytes and one or more target analytes are capable of competitively binding with the at least one antibody. A robotic arm on which the cartridge is disposed is configured to move the cartridge. A pump for introducing the test sample into the at least one opening and removing the test sample from each capillary of the plurality of capillaries is also provided. One or more detectors are also included for measuring respective luminescence levels from each capillary in the plurality of capillaries to determine an amount of the one or more target analytes present in the sample of sweat.

In one aspect, the one or more detectors are part of an imaging module.

In one aspect, automated microfluidic bioreactor further comprises a control system for controlling operation of the robotic arm, pump, and the one or more detectors.

In one aspect, the portion of the plurality of capillaries is a first portion configured to generate at least one first luminescence value and the automated microfluidic bioreactor further comprises a second portion of the plurality of capillaries being comparative capillaries configured to receive a comparative sample having the one or more competitive conjugates, but lacking the target analyte and configured to generate at least one second luminescence level.

In one aspect, the automated microfluidic bioreactor further comprises a processor that is configured to compare the respective measured at least one first luminescence value from the surface of each capillary in the first portion of the plurality of capillaries to a comparative at least one second luminescence value from the surface of each capillary in the second portion of the plurality of capillaries. The processor is configured to determine an amount of the target analyte present in each capillary in the first portion of the plurality of capillaries.

In one aspect, the automated microfluidic bioreactor further comprises a collector for the sample of sweat and a microfluidic channel in fluid communication with the collector, the pump, and the portion of the plurality of capillaries.

In one aspect, the automated microfluidic bioreactor further comprises a reservoir plate comprising the one or more competitive conjugates that receives the sample of sweat optionally comprising the one or more target analytes from the collector, where the test sample is formed.

In one aspect, each capillary of the plurality of capillaries has a respective volume of less than or equal to about 8 μL.

In one aspect, a detection limit of the automated microfluidic bioreactor for the target analyte is less than or equal to about 150 pg/mL.

In one aspect, the test sample comprises a plurality of distinct competitive conjugates respectively capable of binding with a plurality of distinct antibodies and optionally a plurality of distinct target analytes respectively capable of binding with the plurality of distinct antibodies. The plurality of capillaires comprises the plurality of distinct antibodies for competitively binding with the plurality of distinct competitive conjugates or the plurality of target analytes for multiplexed measurement of the plurality of target analytes in the test sample.

In one aspect, at least a portion of the bioreactor microfluidic device is disposable.

In one aspect, a concentration of the at least one antibody on the surface of each capillary of the plurality of capillaries is greater than or equal to about 5 μg/mL to less than or equal to about 25 μg/mL.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1B: FIG. 1A is a schematic of a disposable cartridge used in drug detection prepared in accordance with certain aspects of the present disclosure; FIG. 1B is an illustration of a mechanism of competitive ELISA for drug detection.

FIGS. 2A-2D relate to optimizing an antibody concentration used to coat the capillary inner surface of a disposable cartridge used in drug detection prepared in accordance with certain aspects of the present disclosure. FIG. 2A shows chemiluminescence (CL) intensity (a.u.) versus methadone concentration (ng/mL). Drug concentration varies from 0 to 100 ng/mL that competes with 500× diluted methadone-horseradish peroxidase (HRP). An optimal antibody concentration was determined to be 20 μg/mL. FIG. 2B shows chemiluminescence (CL) intensity (a.u.) versus methamphetamine (METH) concentration (ng/mL). Drug concentration varies from 0 to 1 ng/mL that competes with 2000× diluted METH-HRP. An optimal antibody concentration was determined to be 15 μg/mL. FIG. 2C shows chemiluminescence (CL) intensity (a.u.) versus amphetamine concentration (ng/mL). Drug concentration varies from 0 to 1 ng/mL that competes with 2000× diluted amphetamine-HRP. An optimal antibody concentration was determined to be 10 μg/mL. A slight decrease in the 0.01 ng/mL signal is due to the reason that it is below the detection limit with 2000× diluted amphetamine-HRP. FIG. 2D shows chemiluminescence (CL) intensity (a.u.) versus Tetrahydrocannabinol (THC) concentration (ng/mL). Drug concentration varies from 0 to 1 ng/mL that competes with 2000× diluted THC-HRP. An optimal antibody concentration is determined to be 20 μg/mL. Error bars are obtained with three inter-cartridge measurements.

FIGS. 3A-3D are inhibition curves for four drugs of abuse showing B/Bₒ (%) versus concentration of the respective drugs (ng/mL). FIG. 3A is methadone; FIG. 3B is METH; FIG. 3C is Amphetamine; FIG. 3D is THC.

FIGS. 4A-4C: Dynamic detection ranges in an automated microfluidic bioreactor for detecting one or more drugs prepared in accordance with certain aspects of the present disclosure that comprise methadone (FIG. 4A), methamphetamine (METH) (FIG. 4B), and amphetamine (FIG. 4C). The detection ranges can be shifted to a higher concentration by using a higher concentration of the corresponding competitor conjugate, i.e., drug-HRP. The dilution factors for the drug-HRPs are shown in each of FIGS. 4A-4C. Error bars are obtained from three inter-cartridge measurements.

FIGS. 5A-5C: Multiplexed detection of four drugs, methadone, methamphetamine (METH), amphetamine, and tetrahydrocannabinol (THC) in accordance with certain aspects of the present disclosure. The four drugs were spiked in sweat at high (FIG. 5A), intermediate (FIG. 5B), and low (FIG. 5C) levels of concentrations. The drug concentration is calculated by using the calibration curves in FIGS. 3A-3D and the chemiluminescence signals detected by the capillary sensors. Error bars are obtained with four measurements (see FIG. 7B for the capillary arrangement). The recovery rates are also presented. Table 3 includes the details of the spiked concentrations, calculated concentrations, and the recovery rates.

FIG. 6 shows an example of an automated microfluidic bioreactor device with a cartridge for use in for detecting one or more drugs prepared in accordance with certain aspects of the present disclosure.

FIGS. 7A-7B: FIG. 7A is an illustration of a sensor arrangement to generate inhibition curves for individual drugs (e.g., a single drug) in a cartridge (capillary array) of a microfluidic bioreactor for detecting one or more drugs prepared in accordance with certain aspects of the present disclosure. FIG. 7B is an illustration of a sensor arrangement in a cartridge (capillary array) for multiplexed drug detection (e.g., multiple drugs).

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

Rapid and sensitive drug detection has the potential to guide clinical decision-making on diagnostics and monitoring, as well as patient screening. In various aspects, the present disclosure provides both microfluidic bioreactor devices and methods for rapid, sensitive, and quantitative detection of drugs of abuse in bodily fluids, such as sweat. The method may be conducted to analyze a sample comprising a bodily fluid, such as sweat, in an automated microfluidic system. The method can encompass competitive binding, as well as multiplexed detection of target analytes from the sample. The one or more target analytes potentially present in the fluid sample obtained from the subject may be drug compounds or metabolites of drug compounds of interest. While the present disclosure is directed to detection of drugs of abuse, it will be appreciated that other compounds of interest present in a subject, such as a mammal, may also be tested. As such, the target analyte may be a compound of interest, including drugs of abuse, which may include Schedule I to V drugs categorized by the U.S. Drug Enforcement Administration, pharmaceutical active ingredients, or the like.

The target analytes may be drugs, potential drugs, pharmaceutical active ingredients, or metabolites of these compounds, and may include by way of non-limiting example, controlled substances; narcotics; opioids; performance-enhancing drugs; anti-proliferative agents; anti-rejection drugs; anti-thrombotic agents; anti-coagulants; antioxidants; free radical scavengers; nutrients; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA demethylating agents; cholesterol-lowering agents; vasodilating agents; endogenous vasoactive interference agents; angiogenic substances; cardiac failure active ingredients; targeting toxin agents; and combinations thereof. The description of these target analytes is merely exemplary and should not be considered as limiting as to the scope of compounds that can be detected and quantified in accordance with certain aspects of the present disclosure. Furthermore, various compounds may have various functionalities and thus can be listed in an exemplary class above; however, may be categorized in several different classes of active ingredients.

Various suitable active ingredient or drug compounds are disclosed in Merck Index, An Encyclopedia of Chemicals, Drugs, and Biologicals, Fourteenth Edition (2006) by Merck Research Laboratories and the International Cosmetic Ingredient Dictionary and Handbook, Eleventh Edition (2006) by Cosmetic Toiletry and Fragrance Association, and at http://www.drugbank.ca/, the relevant portions of each of which are incorporated herein by reference. Each additional reference cited or described herein is hereby expressly incorporated by reference in its respective entirety, unless otherwise indicated.

In certain aspects, the one or more drugs (or target analytes) to be detected in accordance with the present disclosure may comprise by way of non-limiting example the following compounds or their metabolites: cannabis or its active cannabinoid compounds (e.g., tetrahydrocannabinol (THC)), methadone, amphetamine, methamphetamine, 3,4-methylenedioxymethamphetamine, cocaine or its active compounds (e.g., benzoylecgonine (BZE)), opiates, heroin, codeine, morphine, hydrocodone, hydromorphone, oxycodone, oxymorphone, 6-acetylmorphine, fentanyl, amphetamine, methamphetamine (METH), methylenedioxymethamphetamine (MDMA), methylenedioxyamphetamine (MDA), phencyclidine (PCP), benzodiazepines, diazepam, alprazolam, anabolic steroids, performance enhancing compounds (e.g., anabolic steroids), synthetic cathinones, synthetic cannabinoids, ethanol, nicotine, caffeine, the like, and combinations thereof.

In certain aspects, the drug detection method may include use of a chemiluminescent enzyme-linked immunosorbent assay (ELISA) or enzyme immunoassay (EIA), in which an antibody is immobilized on a substrate, while the antigen is linked to an enzyme that can complex with the antibody. The conjugated enzyme activity generates chemiluminescence that can be detected and thus can be measured (e.g., semi-quantitatively or quantitatively measured). Due to competitive binding, an amount of analyte in a test sample can then be detected and/or quantified. The detection process can be conducted in a capillary-based immune-biosensor, which may be included in an automated microfluidic bioreactor system provided in accordance with certain aspects of the present disclosure.

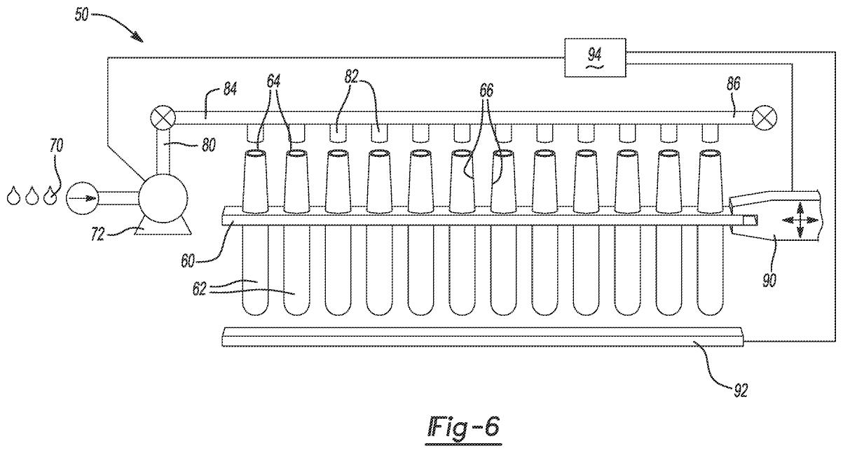

In certain aspects, the disclosure provides an automated microfluidic bioreactor for detecting one or more drugs in a sample of a bodily fluid, such as sweat, from a subject. A simplified example of such an automated microfluidic bioreactor 50 is shown in FIG. 6. As will be appreciated by those of skill in the art, this merely depicts an example of such a device and various other design and configurations may be employed. The bioreactor 50 may comprise a cartridge 60 comprising a plurality of capillaries 62. FIG. 6 shows a disposable cartridge 60 that may be made of a polymer (polystyrene), for example, via an injection molding method. Each cartridge 60 comprises a plurality of capillaries (e.g., at least 2). As shown in FIG. 6, by way of non-limiting example, 12 capillaries, for multiplexed detection of up to 12 analytes.

Each capillary may be a microfluidic capillary optionally having a volume of less than or equal to about 15 μL. In certain variations, each microfluidic capillary may have a volume of less than or equal to about 10 μL, optionally less than or equal to about 9 μL, optionally less than or equal to about 8 μL, optionally less than or equal to about 7 μL, optionally less than or equal to about 6 μL, optionally less than or equal to about 5 μL, and in certain variations, optionally less than or equal to about 4 μL. The microfluidic capillary may have an inner diameter of less than or equal to about 1 mm, optionally having an inner diameter of about 0.8 mm, for example.

In certain aspects, a volume of a test sample of sweat from a subject that is processed in the bioreactor 50 may be less than or equal to about 10 μL, optionally less than or equal to about 9 μL, optionally less than or equal to about 8 μL, optionally less than or equal to about 7 μL optionally less than or equal to about 6 μL, optionally less than or equal to about 5 μL, and in certain variations, optionally less than or equal to about 4 μL The cartridge is mounted on a robotic arm for automated movement. Liquid is withdrawn into or ejected out of the capillaries by an external liquid pump.

Each capillary 62 may have at least one opening 64 and a surface 66 on the interior circumference having at least one antibody (not shown) disposed thereon. While the at least one opening 64 is depicted on the top of each capillary 62, as will be appreciated by those of skill in the art, while not shown, each opening may instead be disposed on a bottom of each capillary 62. In certain aspects, the plurality of capillaries may define an array of capillaries. Each capillary 62 is configured to receive a test sample 70 of sweat comprising one or more competitive conjugates comprising a luminescent moiety and an analyte capable of binding with the at least one antibody. The test sample 70 also comprises one or more target analytes capable of binding with the at least one antibody for participating in a competitive chemiluminescent enzyme-linked immunosorbent assay (ELISA). Notably, while not shown in FIG. 6, the bioreactor 50 may include a sample/reagent reservoir plate or containment vessel to either form and/or store the test sample, which may be sequentially withdrawn into the capillaries 62. The test sample 70 may be formed by adding the competitive conjugates into a collected sample of sweat or other bodily fluid.

A pump 72 may be used for introducing the test sample 70 into the opening 64 from the each capillary 62 and for removing the test sample 70 from each capillary 62 in the array of capillaries. A microfluidic delivery channel or manifold 80 may be connected to the pump 72 and may have one or more channels 82 for fluid communication (injection or suction) with each opening 64 of each capillary 62. As shown, the manifold 80 has a plurality of channels 82; however, as will be appreciated, other configurations may be used, including a single delivery channel 82 that can be passed into respective capillaries 62 as the cartridge 60 is translated. Thus, in certain variations, fluids like the test sample may be selectively introduced into each capillary 62 of the plurality of capillaries. As shown, the manifold 80 has an inlet port 84 and an outlet port 86 so that the test sample 70 can be pumped into and out of the capillaries 62.

The bioreactor 50 may also have a robotic arm 90 on which the cartridge 60 is disposed, which is configured to move and translate the cartridge 60. The robotic arm 90 may also introduce and/or eject reagent or samples. As will be appreciated, the robotic arm 90 may also be a stage or other mechanical component for translating the cartridge 60. One or more detectors 92 are also provided for measuring respective luminescence levels from the surface 66 of each capillary 62 to determine an amount of the one or more target analytes present in the test sample 70 of sweat.

In one aspect, the one or more detectors 92 are part of an imaging module (not shown). In one aspect, the automated microfluidic bioreactor further comprises a control system 94 for controlling operation of the robotic arm 90, pump 72, and the one or more detectors 92. The control system may 94 be a microprocessor or a computer processing unit (CPU). As appreciated by those of skill in the art, the bioreactor 50 may include various other equipment not shown, including flow regulators, valves, gaskets, monitors, and the like.

In one aspect, the automated microfluidic bioreactor includes a processor or CPU that is configured to compare the respective luminescence levels measured by the detector 92 from the surface 66 of each capillary 62 in at least a first portion of the array of capillaries (e.g., test capillaries that provide a first measured luminescence level) to a second comparative luminescence value in a second portion of the array of capillaries (e.g., one or more comparative capillaries) having a comparative surface comprising the at least one antibody contacted with a comparative sample having the one or more competitive conjugates, but lacking the target analyte. Thus, a pair of capillaries can be used to detect each target analyte, where a first capillary is a test capillary that receives the test sample having the potential presence of the target analyte/drug, while the second capillary is a comparative capillary that is free of the target analyte/drug and provides a baseline luminescence level for comparison. Similarly, a baseline or reference level of luminescence may be initially measured from such a comparative capillary and the reference luminescence level then used for comparison to detected and measured luminescence levels in a test capillary. In this manner, an amount of the target analyte present in respective capillaries of the array is determined. The processor may include software that controls the system, reads the chemiluminescence signal (measured luminescence level), and analyzes data.

While not shown in FIG. 6, the automatic microfluidic system may also include a collector module or device, which may be a small accessory used to induce sweat from a subject. Such an integrated sweat collection module can automatically collect and transfer the sweat to the cartridge for detection. For example, the collector can contact a human's finger(s) and raise the temperature of the skin and/or expose the skin to material(s) that can induce sweat. The sweat is collected and transferred via a microfluidic channel to a biochemical reactor attached to the sweat collector, where specific biochemical reaction takes place to quantify the target analytes or drugs of abuse in sweat. The reactor can perform either monoplexed measurement (meaning that it detects only one target analyte at a time) or multiplexed measurement (meaning that it detects multiple distinct target analytes concurrently).

In certain aspects, the total detection time as measured from the time the test sample is introduced into the bioreactor device to the final measurement of the target analyte level is less than or equal to about 30 minutes, in certain aspects, less than or equal to about 20 minutes and in certain variations, optionally be greater than or equal to about 5 minutes to 15 minutes. Further, it is envisioned that smaller cartridges with smaller capillaries can be used to further reduce the sample/reagent consumption.

While detection limits may vary based on various factors, including the nature of the target analyte and antibody used, in certain aspects, the bioreactor may have a detection limit for a target analyte or drug may be less than or equal to about 150 pg/mL, optionally less than or equal to about 145 pg/mL, optionally less than or equal to about 140 pg/mL, optionally less than or equal to about 135 pg/mL, optionally less than or equal to about 130 pg/mL, optionally less than or equal to about 125 pg/mL, optionally less than or equal to about 100 pg/mL, optionally less than or equal to about 75 pg/mL, optionally less than or equal to about 50 pg/mL, optionally less than or equal to about 35 pg/mL, optionally less than or equal to about 25 pg/mL, optionally less than or equal to about 15 pg/mL, optionally less than or equal to about 10 pg/mL, optionally less than or equal to about 5 pg/mL, and in certain variations, optionally less than or equal to about 2 pg/mL.

In certain variations, the automated microfluidic system may have one or more disposable components. This may include a disposable sample/reagent reservoir plate (for sample/reagent storage), a disposable cartridge having an array of capillaries (for ELISA reaction), and/or a disposable collection device. The automated system may further include an automated machine equipped with a liquid pump, a robotic arm, and an imaging module as well as the software that controls the system, reads the chemiluminescence signal, and analyzes data.

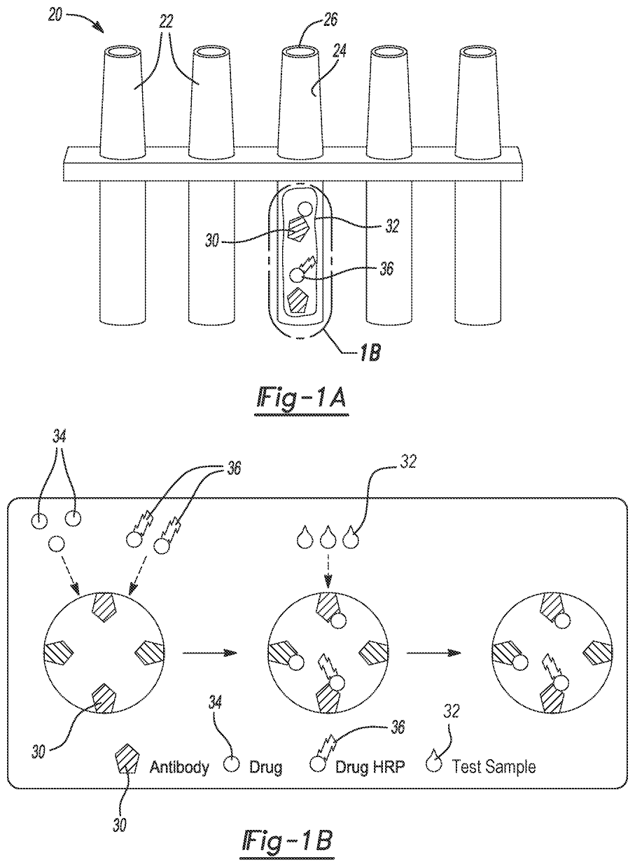

FIG. 1A shows a schematic of a disposable cartridge 20 having a plurality of capillaries 22 used in drug detection according to certain aspects of the present disclosure. FIG. 1B shows the mechanism of competitive ELISA for drug detection that occurs in each capillary 22 of FIG. 1A. The competitive ELISA method for drug detection occurs as follows. Antibody 30 is first coated on the inner surface 24 of at least one capillary 26. The antibody 30 has at least one domain for specifically binding to a target analyte of interest, such as a free drug 34. A test sample 32, which may be a biological sample like sweat, optionally comprises the free drug 34 (i.e., target analyte, whose presence and amount are to be detected) and corresponding drug-HRP conjugate 36 (i.e., competitor or one or more competitive conjugates comprising a luminescent moiety and an analyte) that are mixed and drawn into the capillary 26. In the capillary 26, the free drug 34 and corresponding drug-HRP conjugate 36 compete for limited antibodies 30 on the surface 24. For details of automated device operation, refer to an, Tan, X. et al., “Rapid Mouse Follicle Stimulating Hormone Quantification and Estrus Cycle Analysis Using an Automated Microfluidic Chemiluminescent ELISA System,” ACS Sens.3 (11), pp. 2327-2334 (2018), the relevant portions of which are incorporated herein by reference.

An optimal concentration for antibody for each target analyte is selected to be a relatively low antibody concentration that can generate the largest difference in chemiluminescence signal between the lowest and the highest drug concentrations. By way of example, as will be described further below, an optimal antibody concentration was determined to be 20 μg/mL for methadone, 15 μg/mL for METH, 10 μg/mL for amphetamine, and 20 μg/mL for THC. In certain variations, an antibody concentration on the capillary surface may be greater than or equal to about 5 μg/mL to less than or equal to about 25 μg/mL, optionally greater than or equal to about 10 μg/mL to less than or equal to about 20 μg/mL. For example, the antibody concentration on the surface of the capillary may be 10 μg/mL, 15 μg/mL, or 20 μg/mL in certain variations.

All reagents (e.g., competitive conjugates 36) and sweat test samples 32 may be pre-loaded into a sample/reagent reservoir plate to form a test sample that may be sequentially withdrawn into the capillaries where ELISA reaction(s) occur. Automated cartridge movement, and reagent introduction and ejection are achieved by the robotic arm. One or more signal(s) are recorded through an imaging module after the end of ELISA reaction. In certain aspects, the present disclosure contemplates a competitive enzyme-linked immunosorbent assay (ELISA) where two kinds of molecules (one or more competitive conjugates and one or more target analytes to be detected and quantified) compete to bind on the surface of the capillary having the antibody. This is believed to be the first time that a competitive chemiluminescent enzyme-linked immunosorbent assay (ELISA) has been applied to an automated microfluidic bioreactor system, which provides the capability of detecting drugs (or drug-related metabolites) present in sweat.

As illustrated in FIGS. 1A and 1B, the inner surface 24 of each capillary 22 is first coated with the antibody 30 having a domain configured to bind to a certain analyte/drug 34. Then, mixed solution containing the free drug 34 (i.e., target analyte) and the drug-HRP conjugate 36 (i.e., competitor or competitive conjugate comprising the analyte and a luminescent moiety) compete for the limited amount of antibody 30. With the increased target analyte/drug 34 concentration, the amount of competitive conjugates 36 (e.g., drug-HRP conjugates) bound to the antibody 30 decreases, resulting in a decreased chemiluminescence signal. Thus, a first luminescence level for the test sample that is less than a second luminescence level of a comparative sample free of the drug indicates the presence of the drug. Moreover, the differences in measured luminescence levels can be quantified to provide information about the amount of the drug present in the test sample. The present disclosure demonstrates efficacy with four widely abused drugs, methadone, methamphetamine (METH), amphetamine, and tetrahydrocannabinol (THC). The ability to quantify multiple drugs simultaneously is important in real detection as two or more drugs may be present in sweat of a drug user. Detection of multiplexed drug detection of four illicit substances is demonstrated, as described below.

In certain aspects, the present disclosure relates to a device and a method to rapidly detect target analytes, like drugs of abuse, from human sweat. In certain variations of this device, a small accessory can be used to induce sweat from a subject, such as a human's finger(s) by raising the temperature and/or by exposure to material(s) that can induce sweat. The sweat is collected and transferred via a microfluidic channel to a biochemical reactor attached to the sweat collector, where specific biochemical reaction takes place to quantify the target analytes or drugs of abuse in sweat. The reactor can perform either monoplexed measurement (meaning that it detects only one analyte at a time) or multiplexed measurement (meaning that it detects multiple analytes concurrently).

In certain aspects, the disclosure provides an automated microfluidic bioreactor for detecting one or more drugs in a sample of sweat from a subject. The bioreactor may comprise a cartridge comprising an array of capillaries each comprising at least one opening and a surface having at least one antibody disposed thereon. Each capillary is configured to receive a test sample of sweat comprising one or more competitive conjugates comprising a luminescent moiety and an analyte capable of binding with the at least one antibody. The test sample also comprises one or more target analytes capable of binding with the at least one antibody for participating in a competitive chemiluminescent enzyme-linked immunosorbent assay (ELISA). A robotic arm on which the cartridge is disposed configured to move the cartridge. A pump for introducing the test sample into the opening and removing the test sample from the each capillary of the array of capillaries. One or more detectors is also provided for measuring respective luminescence levels from the surface of each capillary in the array of capillaries to determine an amount of the one or more target analytes present in the sample of sweat.

In one aspect, the one or more detectors are part of an imaging module.

In one aspect, the automated microfluidic bioreactor further comprises a control system for controlling operation of the robotic arm, pump, and the one or more detectors.

In one aspect, the automated microfluidic bioreactor further comprises a processor that is configured to compare the respective measured luminescence levels from the surface of each capillary in the array of capillaries to a comparative luminescence value of a comparative capillary having a comparative surface comprising the at least one antibody contacted with a comparative sample having the one or more competitive conjugates, but lacking the target analyte. In this manner, an amount of the target analyte present in each capillary of the array of capillaries is determined.

In one aspect, the automated microfluidic bioreactor further comprises a collector for the sample of sweat and a microfluidic channel in fluid communication with the collector and with the pump and array of capillaries.

In one further aspect, the automated microfluidic bioreactor comprises a reservoir plate comprising the one or more competitive conjugates that receives the sample of sweat optionally comprising the one or more target analytes from the collector, where the test sample is formed.

In one aspect, each capillary of the array of capillaries has a respective volume of less than or equal to about 8 μL.

In one aspect, a detection limit for the target analyte is less than or equal to about 150 pg/mL, optionally less than or equal to about 100 pg/mL, and for certain target analytes, optionally less than or equal to about 50 pg/mL.

In one aspect, a range of concentrations of the target analyte detected in the test sample of sweat is greater than or equal to about 2 pg/mL to less than or equal to tens of hundreds of ng/mL.

In one aspect, the test sample comprises a plurality of distinct competitive conjugates respectively capable of binding with a plurality of distinct antibodies and optionally a plurality of distinct target analytes respectively capable of binding with the plurality of distinct antibodies, wherein the array of capillaires comprises the plurality of distinct antibodies for competitively binding with the plurality of distinct competitive conjugates or the plurality of target analytes for multiplexed measurement of the plurality of target analytes in the test sample.

The present disclosure also comprises a method of detecting one or more drugs in a sample of sweat from a subject. The method may comprise mixing one or more competitive conjugates comprising a luminescent moiety and an analyte capable of binding with the at least one antibody with a sample of sweat from the subject that optionally comprises a target analyte capable of binding with the at least one antibody to form a test sample. The test sample is introduced into a capillary of a bioreactor microfluidic device having a surface comprising the at least one antibody, so that the test sample contacts the surface to permit competitive binding of the at least one antibody with the one or more competitive conjugates or the target analyte. The method also comprises measuring a first luminescence value from the surface of the capillary after a duration of less than or equal to about 30 minutes after the introducing. The method further comprises comparing the first luminescence value with a second luminescence value of a comparative capillary having a comparative surface comprising the at least one antibody contacted with a comparative sample having the one or more competitive conjugates, but lacking the target analyte, to determine an amount of the target analyte present in the test sample.

In one aspect, the test sample comprises a plurality of distinct competitive conjugates respectively capable of binding with a plurality of distinct antibodies and optionally a plurality of distinct target analytes respectively capable of binding with the plurality of distinct antibodies, wherein the surface of the capillary comprises the plurality of distinct antibodies for competitively binding with the plurality of distinct competitive conjugates or the plurality of target analytes.

In certain aspects, the present disclosure contemplates methods of detecting one or more drugs in a sample of sweat from a subject in a bioreactor microfluidic device, like those described above. The method may include measuring a first luminescence value of a test sample in a bioreactor microfluidic device having a test capillary with a surface comprising at least one antibody, wherein the test sample comprises (i) the sample of sweat from the subject that optionally comprises a target analyte capable of binding with the at least one antibody and (ii) one or more competitive conjugates comprising a luminescent moiety and an analyte capable of binding with the at least one antibody. The measuring occurs by contacting the test sample with the surface to permit competitive binding of the at least one antibody with the one or more competitive conjugates or the target analyte. The measuring occurs within 30 minutes of introducing the test sample into the test capillary. The method further includes comparing the first luminescence value with a second luminescence value of a comparative capillary in the bioreactor microfluidic device having a comparative surface comprising the at least one antibody contacted with a comparative sample having the one or more competitive conjugates, but lacking the target analyte, to determine an amount of the target analyte present in the test sample.

In certain aspects, prior to the measuring, a test sample may be formed by mixing the one or more competitive conjugates comprising the luminescent moiety and the analyte capable of binding with at least one antibody into the sample of sweat obtained from the subject that optionally comprises the target analyte capable of binding with the at least one antibody.

In one aspect, the bioreactor microfluidic device comprises a plurality of capillaries and the introducing comprises selectively introducing the test sample into the plurality of capillaries.

In one aspect, at least a portion of the bioreactor microfluidic device is disposable.

In one aspect, the duration is less than or equal to about 20 minutes and optionally greater than or equal to about 5 minutes to less than or equal to about 15 minutes.

In one aspect, the analyte and the target analyte are the same compound, derivative, or metabolite selected from the group consisting of: cannabinoids, tetrahydrocannabinol (THC), methadone, amphetamine, methamphetamine (METH), 3,4-methylenedioxymethamphetamine, methylenedioxymethamphetamine (MDMA), methylenedioxyamphetamine (MDA), cocaine, benzoylecgonine (BZE), opiates, heroin, codeine, diacetylmorphine, 6-acetylmorphine, morphine, hydrocodone, hydromorphone, oxycodone, oxymorphone, fentanyl, phencyclidine (PCP), benzodiazepines, diazepam, alprazolam, anabolic steroids, performance enhancing compounds, cathinones, ethanol, nicotine, caffeine, and combinations thereof.

Further, a detection limit of the bioreactor microfluidic device for the target analyte is less than or equal to about 150 pg/mL.

In one aspect, the method further comprises collecting the sample of sweat from the subject, wherein the sample of sweat has a total volume of less than or equal to about 4 μL.

The test sample may comprise a plurality of distinct competitive conjugates respectively capable of binding with a plurality of distinct antibodies and optionally a plurality of distinct target analytes respectively capable of binding with the plurality of distinct antibodies. The surface of the capillary comprises the plurality of distinct antibodies for competitively binding with the plurality of distinct competitive conjugates or the plurality of target analytes. The bioreactor microfluidic device comprises a plurality of capillaries and the introducing comprises selectively introducing the test sample into the plurality of capillaries.

In certain aspects, at least a portion of the bioreactor microfluidic device is disposable.

In certain other aspects, a duration of the measuring is less than or equal to about 20 minutes.

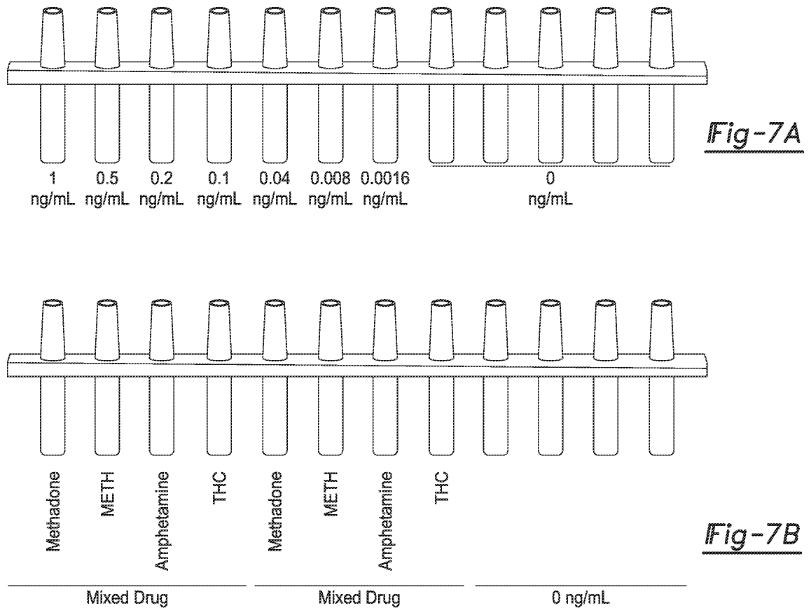

FIGS. 7A-7B contain details of the capillary allocation for drug detection described herein in a cartridge (capillary array) to generate inhibition curves for individual drugs. In FIG. 7A, methadone is used as an example with serial concentrations ranging from 0.016 ng/mL to 1 ng/mL. All twelve capillaries are coated with the antibody against methadone, among which five capillaries are used to generate the blank signal (having a 0 ng/mL concentration of methadone). Three such cartridges are used for each drug to generate the corresponding error bars. In FIG. 7B, a sensor arrangement in a cartridge (capillary array) for multiplexed drug detection is shown. Within a cartridge, two sets of four capillaries, each of which is coated with the corresponding antibody, are allocated to perform duplicate measurement of the same drug mixture. The remaining four capillaries are used to generate the blank signal. For each drug mixture, two such cartridges are used (for a total of four sets of four capillaries). The error bars are obtained from the four sets of measurements.

Here, a rapid tool for detection of drugs of abuse or other target analytes is provided which employs low-volume samples of sweat and such capillary biosensors. The four common illicit drugs, methadone, METH, amphetamine, and THC in artificial sweat, are detected in around 16 minutes and 4 μL sweat is used to achieve the detection limit of 1.6 pg/mL for methadone, 142 pg/mL for METH, 35 pg/mL for amphetamine, and 20 pg/mL for THC. The performance of previous work regarding sample volume, analysis time as well as detection limit is shown and compared in Table 1. Because of low surface-to-volume ratio of conventional wells in a 96-well plate array compared to the microfluidic capillary used in the present disclosure, it is difficult to optimize 96-well plate immunoassay.

TABLE 1
Performance of previous related drug detection work

| | | Analytical | | Sample | Detection | |
|---|---|---|---|---|---|---|
| Drug | Platform | time | | volume | limit | |
| Methadone | Colorimetry | ~1.5 | h | 10 μL | 50 | pg/mL¹ |
| METH | Colorimetry | ~1.5 | h | 10 μL | 1 | ng/mL¹ |
| THC | Colorimetry | ~1.5 | h | 10 μL | 100 | pg/mL¹ |
| Cocaine | Colorimetry | >4 | h | 50 μL | 162 | pg/mL² |
| Morphine | Quantum dots | >1 | h | 100 μL | 270 | pg/mL³ |
| Amphetamine | Colorimetry | >1.5 | h | 25 μL | 10 | ng/mL⁴ |
| Methadone | Colorimetry | >1.5 | h | / | 25 | ng/g⁵ |
| Amphetamine | Colorimetry | >1.5 | h | / | 20 | ng/g⁵ |
| METH | Colorimetry | >1.5 | h | / | 20 | ng/g⁵ |
| METH | Colorimetry | 2 | h | 10 μL | 14.9 | pg/mL⁶ |
| Cocaine | Colorimetry | >50 | min | 25 μL | 60 | pg/mL⁷ |

¹https://toxicology.neogen.com/en/methadone-forensic., the relevant portions of which are incorporated herein by reference.

² Van Der Heide, S.; Calavia, P. G.; Hardwick, S.; Hudson, S.; Wolff, K.; Russell, D. A., A competitive enzyme immunoassay for the quantitative detection of cocaine from banknotes and latent fingermarks. Forensic Sci. Int. 2015, 250, 1-7, the relevant portions of which are incorporated herein by reference.
³ Zhang, C.; Han, Y.; Lin, L.; Deng, N.; Chen, B.; Liu, Y., Development of quantum dots-labeled antibody fluorescence immunoassays for the detection of morphine. J. Agric. Food. Chem. 2017, 65 (6), 1290-1295, the relevant portions of which are incorporated herein by reference.
⁴ Laloup, M.; Tilman, G.; Maes, V.; De Boeck, G.; Wallemacq, P.; Ramaekers, J.; Samyn, N., Validation of an ELISA-based screening assay for the detection of amphetamine, MDMA and MDA in blood and oral fluid. Forensic Sci. Int. 2005, 153 (1), 29-37, the relevant portions of which are incorporated herein by reference.
⁵ Marin, S. J.; Merrell, M.; McMillin, G. A., Drugs of abuse detection in meconium: a comparison between ELISA and biochip microarray. J. Anal. Toxicol. 2011, 35 (1), 40-45, the relevant portions of which are incorporated herein by reference.
⁶ Mao, K.; Yang, Z.; Li, J.; Zhou, X.; Li, X.; Hu, J., A novel colorimetric biosensor based on non-aggregated Au@ Ag core-shell nanoparticles for methamphetamine and cocaine detection. Talanta 2017, 175, 338-346, the relevant portions of which are incorporated herein by reference.
⁷ Vidal, J. C.; Bertolín, J. R.; Bonel, L.; Asturias, L.; Arcos-Martínez, M. J.; Castillo, J. R., Rapid determination of recent cocaine use with magnetic particles-based enzyme immunoassays in serum, saliva, and urine fluids. J. Pharm. Biomed. Anal. 2016, 125, 54-61, the relevant portion of which are incorporated herein by reference.

### EXAMPLE 1

In one example, the target analytes may include methadone, methamphetamine (METH), amphetamine, and tetrahydrocannabinol (THC) spiked in artificial sweat as a model system. Rapid and quantitative detection with approximately 4 μL of sweat sample per analyte in approximately 16 minutes is demonstrated. The results illustrate a dynamic range of 0.0016 ng/mL-1 ng/mL for methadone, 0.016 ng/mL-25 ng/mL for METH, 0.005 ng/mL-10 ng/mL for amphetamine, and 0.02 ng/mL-1000 ng/mL for THC. Cross-activity among four drugs is also determined. The present disclosure thus provides promising strategy for rapid and on-site sweat detection and multi-drug analysis.

Reagents

Methadone antibody (20-1576), methamphetamine antibody (10-2731), amphetamine antibody (10-1509), tetrahydrocannabinol antibody (10-T43E), methadone-horseradish peroxidase (methadone-HRP, 80-1208—a chemiluminescent moiety), methamphetamine-HRP (65-IM52—a chemiluminescent moiety), amphetamine-HRP (65-IA52—a chemiluminescent moiety), and tetrahydrocannabinol-HRP (65-IT60—a chemiluminescent moiety) were purchased from Fitzgerald. Methadone solution in methanol (M007), methamphetamine solution in methanol (M009), amphetamine solution in methanol (A007), and tetrahydrocannabinol solution in methanol (T4764) were bought from Sigma-Aldrich. ELISA plate-coating buffer (1× PBS, DY006), reagent diluent concentrate (10% BSA in 10× PBS), and Quantikine ELISA wash buffer (25×, WA126) were purchased from R&D Systems. The chemiluminescent substrate (SuperSignal ELISA Femto Substrate, 37074) was purchased from Thermo Fisher. Artificial eccrine perspiration (pH=4.5, 1700-0020) was bought from Pickering Laboratories.

Capillary Based Immune-Biosensor

The automated microfluidic system comprises a disposable sample/reagent reservoir plate (for sample/reagent storage), a disposable cartridge having a capillary array (for ELISA reaction), and an automated machine equipped with a liquid pump, a robotic arm, and an imaging module as well as the software that controls the system, reads the chemiluminescence signal, and analyzes data. The disposable cartridge was made of polystyrene through the injection molding method (see FIG. 6). Each cartridge contained 12 capillaries for multiplexed detection of up to 12 analytes (see FIG. 1A). Each capillary had an inner diameter of 0.8 mm and a volume of 8 μL. During the experiment, all reagents and sweat samples were pre-loaded into the sample/reagent reservoir plate and were sequentially withdrawn into the capillaries where ELISA reaction occurred. Automated cartridge movement, and reagent introduction and ejection were achieved by the robotic arm. Signal was recorded through an imaging module after the end of ELISA reaction. In this study, competitive enzyme-linked immunosorbent assay (ELISA) where two kinds of molecules competed to bind on the capillary was applied to the microfluidic system for the first time. Besides, quantifying multiple drugs simultaneously is important in real detection as two or more drugs may be present in sweat of a drug user. Here, multiplexed drug detection of four illicit substances is demonstrated.

Drug Detection Protocols

Competitive ELISA reactions are used to detect drugs (or drug-related metabolites) in sweat. As illustrated in FIGS. 1A-1B, the inner surface of each capillary was first coated with the antibody for a certain analyte. Then, mixed solution containing the free drug (i.e., analyte) and the drug-HRP conjugate (i.e., competitor) competed for the limited amount of antibody. With the increased analyte concentration, the amount of drug-HRP conjugates bound to the antibody decreases, resulting in a decreased chemiluminescence signal. In the current study, four widely abused drugs, methadone, METH, amphetamine, and THC were used as the model system.

Preparation

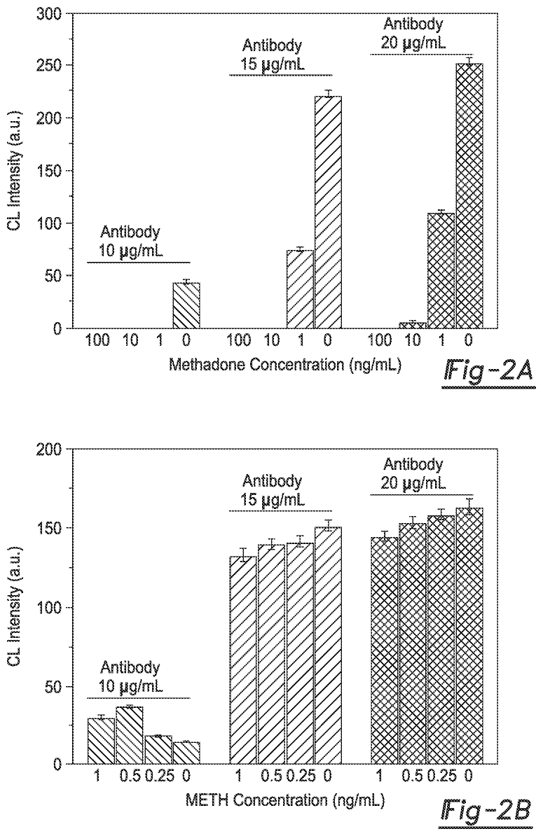

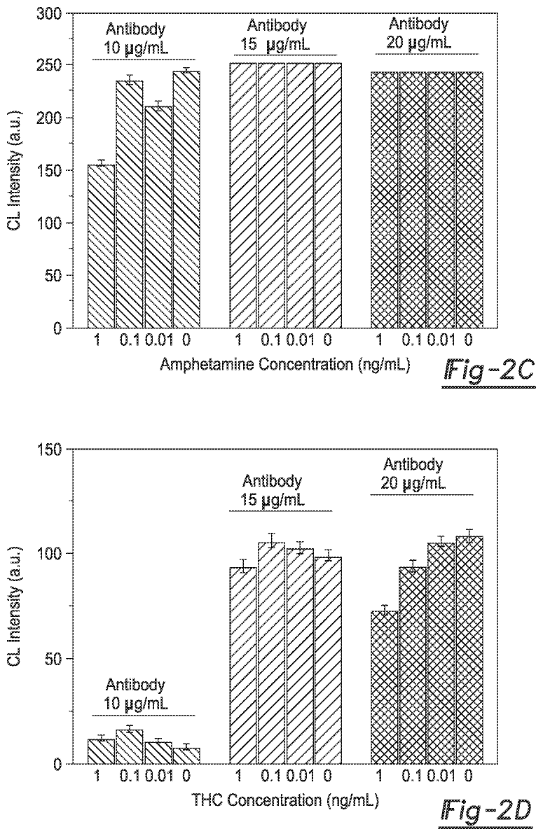

The capillary inner surface was coated with the corresponding antibody by incubating the antibody diluted with ELISA plate-coating buffer inside the capillary for 1 hour, followed by washing with 1× wash buffer for 20 s. The concentration of the antibody used to coat the sensor surface can be optimized. Insufficient antibody concentration leads to a low antibody surface density, which, in turn, causes an uneven antibody distribution and a decrease in detection signal (see, for example, FIGS. 2A, 2B, and 2D). On the other hand, in the presence of excessive antibody on the surface, drug and drug-HRP bind to it without competition, which deteriorates the detection sensitivity and limits the dynamic range (i.e., it makes the signal to saturate easily—see FIG. 2C, for example). Therefore, the optimal concentration for antibody for each target analyte is selected to be the lowest possible antibody concentration (in order to save the antibody) that can generate the largest difference in chemiluminescence signal between the lowest and the highest drug concentration. As shown in FIGS. 2A-2D, the optimal antibody concentration was determined to be 20 μg/mL for methadone, 15 μg/mL for METH, 10 μg/mL for amphetamine, and 20 μg/mL for THC.

After antibody incubation, the capillaries were blocked with 1× reagent diluent (1% BSA in 1× PBS) for 40 minutes at room temperature. Finally, washing was conducted again to remove unbound molecules. Solutions containing the analytes (i.e., free drug molecules) were diluted with artificial sweat in order to prepare free drug standards at serial concentrations. All reagents such as drug-HRP conjugates, wash buffer, and substrate were pre-loaded into the wells on the sample/reagent reservoir plate.

Drug Detection

Actual drug detection was performed as follows. (1) An equal volume of free drug standards was manually mixed with drug-HRP solutions of pre-determined concentrations, which competed with the free drug molecules in binding to the antibodies on the capillary surface, in wells of the sample/reagent reservoir plate. (2) 8 μL of mixture solution was drawn into the capillary and incubated inside the capillary for 15 minutes. (3) The solution was ejected from the capillary to the waste well on the sample/reagent reservoir plate, followed by rinse with 1× wash buffer twice (20 seconds for each wash). (4) The chemiluminescent substrate was withdrawn into the capillaries and the chemiluminescent images were recorded by a CMOS camera after 3 seconds of incubation. The entire detection time (i.e., from loading samples to recording results) was approximately 16 minutes. The sensor arrangement in a cartridge to detect each individual drug is illustrated in FIG. 7A.

Signal Analysis

ImageJ software was used to analyze the images. Blue chemiluminescence was extracted and its intensity counts along the capillary longitudinal direction was recorded and averaged among 150 pixels (about 3.7 mm in length along the capillary center line), which was related to free drug concentrations via the inhibition curve (IC), i.e., the inhibition ratio B/B₀(%) as a function of the analyte (free drug) concentration, where B is the signal of a drug at a certain concentration and B₀ is the signal of the drug at zero concentration.

Characterization of Cross-Reactivity

In order to develop a sensor array that can quantify the presence of multiple analytes simultaneously, it is important to examine their cross-reactivity. Using the methadone as an example, methadone was the target analyte and channels of the capillary sensor were first coated with methadone antibody. After blocking, serial concentrations of the challenging analytes, METH, amphetamine, or THC, were individually mixed with methadone-HRP and withdrawn into the capillaries. The challenging analyte molecules (e.g., METH) competed with methadone-HRP for the methadone antibody coated on the capillary surface. Cross-reactivity is calculated as follows:

Cross-reactivity (%)=[IC(methadone)so]/[IC(challenging analyte)₅₀]×100%, (1) where [IC₅₀] is the half-maximum inhibition concentration.

Multiplexed Detection of Drugs

In order to demonstrate multiplexed drug detection capability, four drugs at three different levels (high, intermediate, and low concentrations) were spiked into sweat. Four capillaries on a cartridge coated with four different antibodies were used to detect the four drugs. Four drug-HRP conjugates were prepared individually at 2000×, 4000×, 15000×, and 4000× dilution for methadone-HRP, METH, amphetamine-HRP, and THC-HRP, respectively, for the detection of the corresponding drugs. The sensor arrangement in a cartridge for multiplexed drug detection is illustrated in FIG. 7B.

Furthermore, to improve convenience in practical utility, individual drug-HRP was replaced with a mixture of drug-HRPs with the final concentration equivalent to 2000×, 4000×, 15000×, and 4000× dilution for methadone-HRP, METH-HRP, amphetamine-HRP, and THC-HRP, respectively. The remaining procedures were the same as the experiments in the multiplexed drug detection described previously.

Detection of Individual Drugs

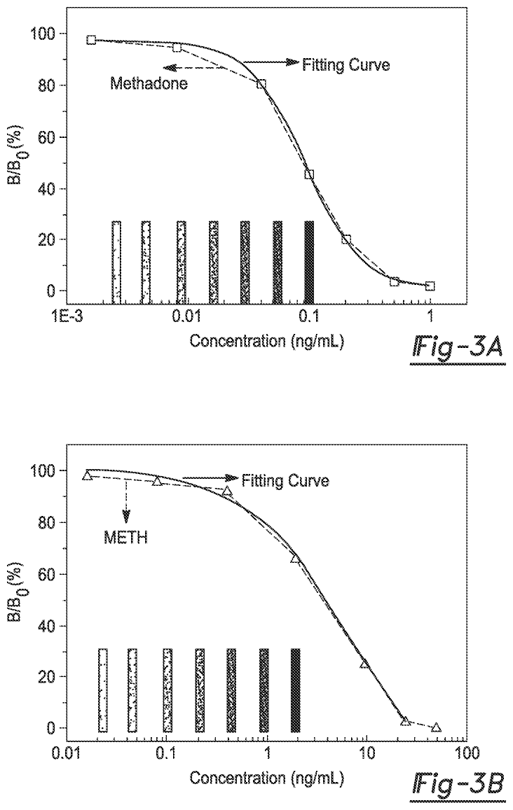

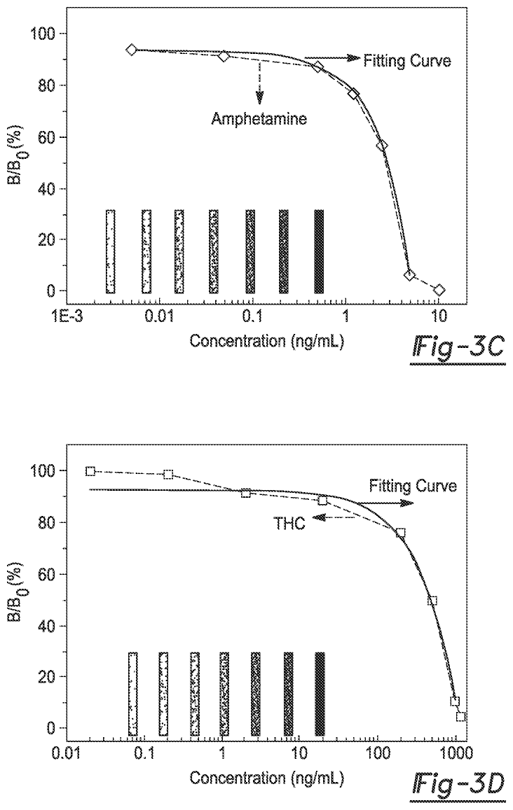

The sensing performance of the device in drug detection was systematically investigated. Each individual drug with various concentrations in sweat was analyzed and the entire analysis took approximately 16 minutes to complete. The capillary arrangement is illustrated in FIG. 7A. Three cartridges were used for each drug to generate the error bars. The corresponding inhibition curve is plotted in FIGS. 3A-3D.

In FIG. 3A, the inhibition curve for methadone is shown. Dynamic range: 0.0016 ng/mL-1 ng/mL. In FIG. 3B, the inhibition curve for methamphetamine (METH) is shown. Dynamic range: 0.016 ng/mL-25 ng/mL. In FIG. 3C, the inhibition curve for amphetamine is shown. Dynamic range: 0.005 ng/mL-10 ng/mL. In FIG. 3D, the inhibition curve for tetrahydrocannabinol (THC) is shown. Dynamic range: 0.02 ng/mL-1000 ng/mL. Solid black lines are the logistic curve fit. Error bars are obtained with three inter-cartridge measurements. Insets show the exemplary images for drugs of various concentrations, from which the chemiluminescence signal are extracted. The concentration of the competitor was 2000×, 4000×, 15000×, and 4000× dilution from the original concentration provided by the vendor (Fitzgerald) for methadone-HRP, METH-HRP, amphetamine-HRP, and THC-HRP, respectively.

Averaged relative standard deviation is 0.6% for methadone, 1.4% for METH, 0.4% for amphetamine, and 1.0% for THC, showing good reproducibility among cartridges. Due to the competitive nature of the analyte and its competitor (i.e., drug-HRP), the higher the analyte concentration, the lower the chemiluminescence signal. In general, all inhibition curves follow a logistic relationship with R²>0.99 (x is in the units of ng/mL):

y=0.87+96.23/(1+(x/0.09)^{1.87}) for methadone;

y=−35.27+136.48/(1+(x/7.63)^{0.81}) for METH;

y=−1.31+94.46/(1+(x/1473.04)^{1.2} ) for amphetamine, and

y=−2.14+93.79/(1+(x/5.95)^{0.92}) for THC.

These curves will be used as a calibration curve in the subsequent tests. With the above inhibition curves, the detection limit (IC_{(100-3δ)}, δ is the standard deviation of artificial sweat without analyte and is 0.05%, 1.3%, 2.4%, and 2.8% for methadone, METH, amphetamine , and THC, respectively) is estimated to be 1.6 pg/mL for methadone, 142 pg/mL for METH, 35 pg/mL for amphetamine, and 20 pg/mL for THC, indicating the high sensitivity of the device (while being rapid), which are similar to or even better than commercially available products and previous work, which are usually on the order of 100-1,000 pg/mL.

Tuning of the Dynamic Range

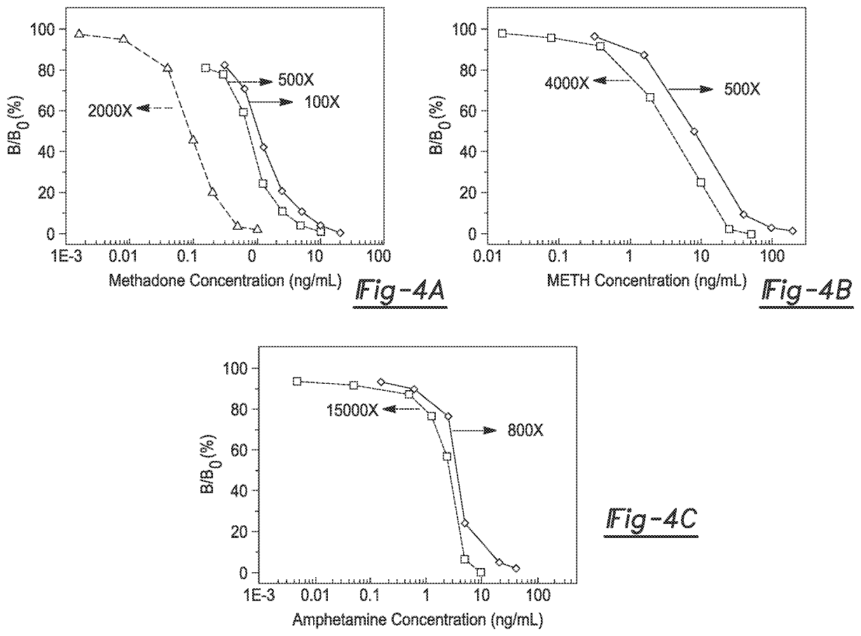

Since many drugs of abuse have been legalized in many states in the U.S. (and in Canada), the presence of drugs and their corresponding metabolites in sweat of a drug user does not necessarily establish the case of illegal drug use—it all depends on the appropriate cut-off. For example, the typical concentration of a drug in sweat of someone who uses a drug can be as large as greater than 10 ng/mL. Therefore, it is important that the dynamic range of a sensor be adjustable to cover both sides of the cut-off values, should those cut-off values be established. FIGS. 4A-4C show that the dynamic range of the drugs (especially methadone, METH, and amphetamine) can be tuned by changing the concentration of the corresponding competitor (i.e., drug-HRP). With the increased concentration of drug-HRP, the entire inhibition curve shifts to a higher drug concentration range. This is because a higher concentration of the drug is needed to compete with the higher concentration of drug-HRP in order to generate the same chemiluminescence signal. As shown in FIGS. 4A-4C, methadone concentration up to 1 ng/mL, 10 ng/mL, and 20 ng/mL can be quantitatively analyzed with 2000×, 500×, and 100× dilution of methadone-HRP, respectively. Similarly, the upper limit of METH is shifted from 25 ng/mL to 200 ng/mL with 8× higher METH-HRP concentration, and the upper limit of amphetamine is shifted from 10 ng/mL to 40 ng/mL by changing the dilution factor of amphetamine-HRP from 15000× to 8000×. Note that the detection range of THC is sufficiently wide (see FIG. 3D) and therefore does not need any further adjustment.

Cross-Reactivity of Capillary Based Immunoassay Sensor

In order to perform multiplexed detection of drugs in sweat, first the cross-reactivity of the capillary-based sensors is examined. The capillary coated with the antibody intended for detecting one particular drug (i.e., target analyte) is challenged with various concentrations of the other three drugs (i.e., challenging analytes). Here, the concentrations of 10,000 ng/mL, 5,000 ng/mL, 400 ng/mL, 80 ng/mL, and 1.6 ng/mL for the challenging analytes in sweat are used. The cross-reactivity is calculated according to Eq. (1) and the corresponding results are presented in Table 2. Overall, the sensors exhibit excellent specificity with the cross-reactivity far less than 1%. One exception is the 3.1% cross-reactivity of METH sensor to amphetamine, which is due to the structural similarities between METH and amphetamine.

TABLE 2
Cross-reactivity of four drug sensors

| Target analyte | Challenging analyte | Cross-reactivity (100%) |
|---|---|---|
| Methadone | METH | <0.001% |
| | Amphetamine | <0.001% |
| | THC | <0.001% |
| METH | Methadone | <0.04% |
| | Amphetamine | 3.1% |
| | THC | <0.04% |
| Amphetamine | Methadone | <0.02% |
| | METH | 0.07% |
| | THC | <0.02% |
| THC | Methadone | <0.4% |
| | METH | <0.4% |
| | Amphetamine | <0.4% |

The final concentration of competing drug conjugates was 2000×, 4000×, 15000×, and 4000× for methadone-HRP, METH-HRP, amphetamine-HRP, and THC-HRP, respectively, in corresponding sensors.

### EXAMPLE 2

Multiplexed Drug Detection

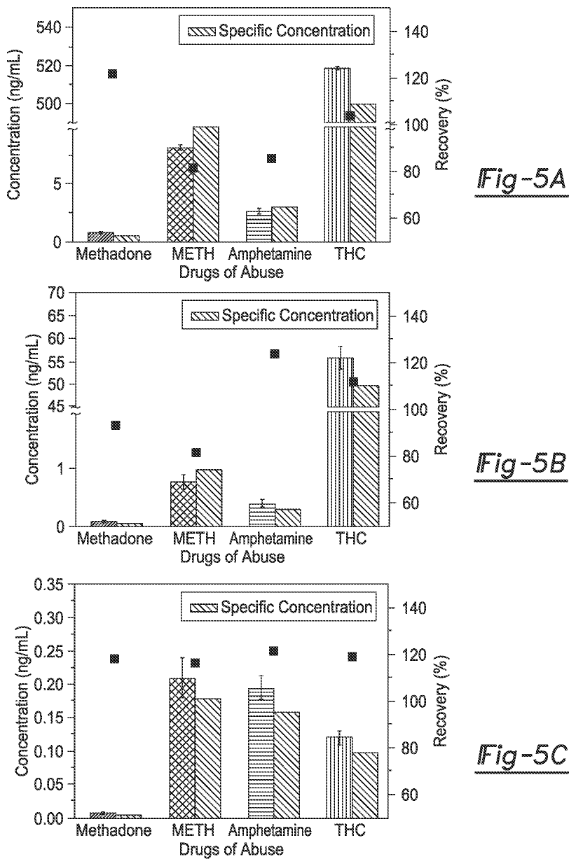

In the Example above, each individual drug was added to sweat and analyzed by the microfluidic bioreactor sensor. In reality, multiple drugs (and the associated metabolites) may be present in sweat of a drug user. Therefore, it is important to quantify multiple drugs in sweat simultaneously. To demonstrate the multiplexed drug detection capability of the sensors, a drug mixture containing all four drugs was prepared to compete with individual drug-HRP conjugate for the detection of the corresponding drugs (see the arrangement of the capillary array in FIG. 7B). By using the chemiluminescence signal and the calibration curves in FIGS. 3A-3D, the concentration of a target drug in sweat can be quantified. The quantification and the recovery rate for each drug are shown in FIGS. 5A-5C and Table 3. Overall, the recovery rate (defined as the ratio between the observed and spiked concentration) is between 75% and 120% with an average of 106% among all concentration levels, which proves the quantitative analysis capability and the reliability of the sensors. Furthermore, the drug detection is performed by using the mixed drug solution with one drug absent in order to confirm the cross-reactivity. The corresponding results are shown in Table 4. Again, no cross-reactivity was observed (i.e., the sensors reported zero concentrations for the missing drugs).

TABLE 3
Recovery rate of multiplexed drug detection

| | | | | | Averaged |
|---|---|---|---|---|---|
| | | Spiked | Calculated | Recovery rate | recovery |
| | Drug | (ng/mL) | (ng/mL) | (%) | rate (%) |
| High level | Methadone | 0.5 | 0.61 ± 0.05 | 122.0 | 98 |
| | METH | 10 | 8.12 ± 0.22 | 81.2 | |
| | Amphetamine | 3 | 2.56 ± 0.31 | 85.2 | |
| | THC | 500 | 518.40 ± 1.01 | 103.7 | |
| Intermediate | Methadone | 0.05 | 0.047 ± 0.03 | 93.6 | 101.2 |
| level | METH | 1 | 0.76 ± 0.13 | 75.6 | |
| | Amphetamine | 0.3 | 0.37 ± 0.08 | 123.6 | |
| | THC | 50 | 55.9 ± 2.48 | 111.8 | |
| Low level | Methadone | 0.006 | 0.007 ± 0.002 | 118.3 | 118.9 |
| | METH | 0.18 | 0.21 ± 0.03 | 116.6 | |
| | Amphetamine | 0.16 | 0.19 ± 0.02 | 121.3 | |
| | THC | 0.1 | 0.12 ± 0.01 | 119.3 | |

TABLE 4
Recovery rate of multiplexed drug detection with one drug absent

| | | Excepted | | | Averaged |
|---|---|---|---|---|---|
| | | (Spiked) | Observed | Recovery | recovery |
| | Drug | (ng/mL) | (ng/mL) | rate (%) | rate (%) |
| High level | Methadone | 0.6 | 0.61 ± 0.14 | 101.9 | 91.7 |
| | METH | 18 | 14.60 ± 2.83 | 81.0 | |
| | Amphetamine | 0 | 0 | — | |
| | THC | 800 | 738 ± 5.70 | 92.2 | |
| Intermediate | Methadone | 0.06 | 0.07 ± 0.03 | 110.3 | 109.0 |
| level | METH | 0 | 0 | — | |
| | Amphetamine | 1.6 | 1.77 ± 0.38 | 110.6 | |
| | THC | 100 | 106.20 ± 2.92 | 106.2 | |
| Low level | Methadone | 0 | 0 | — | 119.1 |
| | METH | 0.18 | 0.21 ± 0.03 | 116.6 | |
| | Amphetamine | 0.16 | 0.20 ± 0.02 | 121.3 | |
| | THC | 0.1 | 0.12 ± 0.01 | 119.3 | |
| Intermediate | Methadone | 0.5 | 0.61 ± 0.05 | 122.0 | 96.1 |
| level | METH | 10 | 8.12 ± 0.22 | 81.2 | |
| | Amphetamine | 3 | 2.56 ± 0.31 | 85.2 | |
| | THC | 0 | 0 | — | |

In the experiment above, the drug-HRP conjugate was prepared individually for each target drug to be detected. In practice, it is more convenient and cost-effective to prepare all drug-HRP conjugates in one solution so that only one storage chamber (or vial) is needed instead of four in an eventual device. To test the feasibility, the mixture of drug-HRP conjugates containing all four competitors is prepared to compete with drug mixture that contained all four drugs. Again, by using the chemiluminescence signal and the calibration curves in FIGS. 3A-3D, the drug concentration in sweat was quantified. The quantification and the recovery rate for each drug are shown in Table 5. Some individual recovery rates can be as low as 52% (for METH) and as high as 180% for THC, suggesting that the mixed drug-HRP conjugate method can be further improved for comprehensive quantitative analysis of drugs.

TABLE 5
Recovery rate of multiplexed drug detection with mixed drug-HRP conjugates

| | | Excepted | | | Averaged |
|---|---|---|---|---|---|
| | | (Spiked) | Observed | Recovery | recovery |
| | Drug | (ng/mL) | (ng/mL) | (%) | rate (%) |
| High level | Methadone | 0.5 | 0.35 ± 0.06 | 70.0 | 71.9 |
| | METH | 10 | 5.23 ± 1.27 | 52.3 | |
| | Amphetamine | 3 | 2.17 ± 0.31 | 72.3 | |
| | THC | 500 | 463.70 ± 4.90 | 92.8 | |
| Intermediate | Methadone | 0.05 | 0.06 ± 0.02 | 127.4 | 125.7 |
| level | METH | 1 | 1.43 ± 0.32 | 142.5 | |
| | Amphetamine | 0.3 | 0.41 ± 0.11 | 135.3 | |
| | THC | 50 | 48.80 ± 3.54 | 97.6 | |
| Low level | Methadone | 0.006 | 0.01 ± 0.01 | 118.3 | 139.8 |
| | METH | 0.18 | 0.24 ± 0.03 | 133.2 | |
| | Amphetamine | 0.16 | 0.20 ± 0.02 | 127.5 | |
| | THC | 0.1 | 0.18 ± 0.03 | 180.0 | |

Compared to other biological sources, like blood and hair, sweat illustrates distinctive characteristics of being easily accessible and non-invasive as the ideal drug detection candidate. Immunoassays based on 96-well plates have been applied as one of primary tools for drug detection in vitro. It is simple and does not require expensive instruments. Besides, many labels such as fluorescence, colorimetry, quantum dots, and chemiluminescence, are developed and optimized to increase the detection sensitivity. For example, the quantitative detection of cocaine through competitive enzyme immunoassay in a 96-well plate has been described in Van Der Heide, S. et al.,“A competitive enzyme immunoassay for the quantitative detection of cocaine from banknotes and latent fingermarks,” Forensic Sci. Int. 250, pp. 1-7 (2015). A limit of detection of 162 pg/mL was achieved, which was comparable to that of conventional gas chromatography/liquid chromatography-mass spectrometry techniques. However, the whole assay took more than 4 hours and 100 μL of a costly reagent was applied in each procedure. A study based on quantum dots-labeled antibody fluorescence immunoassays for the detection of morphine shortened the detection time to 1 hour, but the detection limit of 270 pg/mL still needed improvement. Some commercial products from Neogen Corporation (United States) further improves drug detection sensitivity to about 1.5 hours.

Thus, the inventive methods of detecting one or more drugs in a sample (e.g., of sweat) from a subject provide sensitive and quantitative techniques for rapid and on-site detection of multiple drugs of abuse using a microfluidic capillary based sensor array by using a competitive ELISA protocol. The present disclosure shows that the microfluidic bioreactor device is able to complete the assay of four drugs in sweat in approximately 16 minutes with only 4 μL of sweat for each drug. The detection range is between a few pg/mL to tens-of-hundreds of ng/mL and can be tuned for different applications. In addition, the detection range can be tuned for different applications by adjusting the competitor conjugates' concentrations. The present disclosure thus contemplates an autonomous, portable, and cost-effective device for hospital testing, workplace drug-use screening, roadside testing, and patient monitoring in drug rehabilitation centers, among others.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method of detecting one or more drugs in a sample of sweat from a subject, the method comprising:
measuring a first luminescence value of a test sample in a bioreactor microfluidic device having a test capillary with a surface comprising at least one antibody, wherein the test sample comprises (i) the sample of sweat from the subject that optionally comprises a target analyte capable of binding with the at least one antibody and (ii) one or more competitive conjugates comprising a luminescent moiety and an analyte capable of binding with the at least one antibody, by contacting the test sample with the surface to permit competitive binding of the at least one antibody with the one or more competitive conjugates or the target analyte, wherein the measuring occurs within 30 minutes of introducing the test sample into the test capillary; and
comparing the first luminescence value with a second luminescence value of a comparative capillary in the bioreactor microfluidic device having a comparative surface comprising the at least one antibody contacted with a comparative sample having the one or more competitive conjugates, but lacking the target analyte, to determine an amount of the target analyte present in the test sample.

2. The method of claim 1, further comprising prior to the measuring, forming a test sample by mixing the one or more competitive conjugates comprising the luminescent moiety and the analyte capable of binding with at least one antibody into the sample of sweat obtained from the subject that optionally comprises the target analyte capable of binding with the at least one antibody.

3. The method of claim 1, further comprising collecting the sample of sweat from the subject, wherein the sample of sweat has a total volume of less than or equal to about 4 μL.

4. The method of claim 1, wherein the test sample comprises a plurality of distinct competitive conjugates respectively capable of binding with a plurality of distinct antibodies and optionally a plurality of distinct target analytes respectively capable of binding with the plurality of distinct antibodies, wherein the surface of the capillary comprises the plurality of distinct antibodies for competitively binding with the plurality of distinct competitive conjugates or the plurality of target analytes.

5. The method of claim 1, wherein the bioreactor microfluidic device comprises a plurality of capillaries and the introducing comprises selectively introducing the test sample into the plurality of capillaries.

6. The method of claim 1, wherein at least a portion of the bioreactor microfluidic device is disposable.

7. The method of claim 1, wherein a duration of the measuring is less than or equal to about 20 minutes.

8. The method of claim 1, wherein the analyte and the target analyte are the same compound, derivative, or metabolite selected from the group consisting of: cannabinoids, tetrahydrocannabinol (THC), methadone, amphetamine, methamphetamine (METH), 3,4-methylenedioxymethamphetamine, methylenedioxymethamphetamine (MDMA), methylenedioxyamphetamine (MDA), cocaine, benzoylecgonine (BZE), opiates, heroin, codeine, diacetylmorphine, 6-acetylmorphine, morphine, hydrocodone, hydromorphone, oxycodone, oxymorphone, fentanyl, phencyclidine (PCP), benzodiazepines, diazepam, alprazolam, anabolic steroids, performance enhancing compounds, cathinones, ethanol, nicotine, caffeine, and combinations thereof.

9. The method of claim 1, wherein a detection limit of the bioreactor microfluidic device for the target analyte is less than or equal to about 150 pg/mL.

10. An automated microfluidic bioreactor for detecting one or more drugs in a sample of sweat from a subject, the bioreactor comprising:
a cartridge comprising a plurality of capillaries wherein each capillary comprises at least one opening and a surface having at least one antibody disposed thereon for participating in a competitive chemiluminescent enzyme-linked immunosorbent assay (ELISA), wherein a portion of the plurality of capillaries is configured to receive a test sample comprising sweat from the subject, one or more competitive conjugates comprising a luminescent moiety and one or more analytes, and optionally one or more target analytes, wherein the one or more analytes and one or more target analytes are capable of competitively binding with the at least one antibody;
a robotic arm on which the cartridge is disposed configured to move the cartridge;
a pump for introducing the test sample into the at least one opening and removing the test sample from each capillary of the plurality of capillaries; and
one or more detectors for measuring respective luminescence levels from each capillary in the plurality of capillaries to determine an amount of the one or more target analytes present in the sample of sweat.

11. The automated microfluidic bioreactor of claim 10, wherein the one or more detectors are part of an imaging module.

12. The automated microfluidic bioreactor of claim 1, further comprising a control system for controlling operation of the robotic arm, pump, and the one or more detectors.

13. The automated microfluidic bioreactor of claim 1, wherein the portion of the plurality of capillaries is a first portion configured to generate at least one first luminescence value and the automated microfluidic bioreactor further comprising a second portion of the plurality of capillaries being comparative capillaries configured to receive a comparative sample having the one or more competitive conjugates, but lacking the target analyte and configured to generate at least one second luminescence level.

14. The automated microfluidic bioreactor of claim 13, further comprising a processor that is configured to compare the respective measured at least one first luminescence value from the surface of each capillary in the first portion of the plurality of capillaries to a comparative at least one second luminescence value from the surface of each capillary in the second portion of the plurality of capillaries, wherein the processor is configured to determine an amount of the target analyte present in each capillary in the first portion of the plurality of capillaries.

15. The automated microfluidic bioreactor of claim 1, further comprising a collector for the sample of sweat and a microfluidic channel in fluid communication with the collector, the pump, and the portion of the plurality of capillaries.

16. The automated microfluidic bioreactor of claim 15, further comprising a reservoir plate comprising the one or more competitive conjugates that receives the sample of sweat optionally comprising the one or more target analytes from the collector, where the test sample is formed.

17. The automated microfluidic bioreactor of claim 1, wherein each capillary of the plurality of capillaries has a respective volume of less than or equal to about 8 μL.

18. The automated microfluidic bioreactor of claim 1, wherein a detection limit of the automated microfluidic bioreactor for the target analyte is less than or equal to about 150 pg/mL.

19. The automated microfluidic bioreactor of claim 1, wherein the test sample comprises a plurality of distinct competitive conjugates respectively capable of binding with a plurality of distinct antibodies and optionally a plurality of distinct target analytes respectively capable of binding with the plurality of distinct antibodies, wherein the plurality of capillaires comprises the plurality of distinct antibodies for competitively binding with the plurality of distinct competitive conjugates or the plurality of target analytes for multiplexed measurement of the plurality of target analytes in the test sample.

20. The automated microfluidic bioreactor of claim 1, wherein at least a portion of the bioreactor microfluidic device is disposable.

21. The automated microfluidic bioreactor of claim 1, wherein a concentration of the at least one antibody on the surface of each capillary of the plurality of capillaries is greater than or equal to about 5 μg/mL to less than or equal to about 25 μg/mL.
